(19) United States
(12) Patent Application Publication
(10) Pub. No.: US 2011/0313315 A1
(43) Pub. Date: Dec. 22, 2011

(54) AUDITORY DIAGNOSIS AND TRAINING SYSTEM APPARATUS AND METHOD

(76) Inventors: Joseph Attias, Petach-Tikva (IL); Rafi Shemesh, Holon (IL)

(21) Appl. No.: 13/147,284

(22) PCT Filed: Feb. 1, 2010
(86) PCT No.: PCT/IL10/00087

§ 371 Date: Aug. 24, 2011

Related U.S. Application Data
(60) Provisional application No. 61/149,083, filed on Feb. 2, 2009.

(51) Int. Cl. A61B 5/00

(52) U.S. Cl. 600/559

(57) ABSTRACT

The present invention provides a system and method for auditory skills Improvement and, more specifically, for improving auditory perception using a system and method that screens, diagnoses and trains the auditory system. The system is compatible for hearing aid users and for children. The training is specific to the auditory profile of the patients with different task difficulties. To monitor and control the training, the system transfer specific data to a remote server.

### FIELD OF THE INVENTION

The invention relates generally to the field of auditory skills improvement and, more specifically, to improving auditory perception using an automatic and interactive system that screens, diagnoses and trains the auditory system.

### BACKGROUND OF THE INVENTION

Hearing loss is one of the most prevalent chronic health impairments affecting both adults and children. There are about 300 million people world wide with hearing impairment. However, only 20% of them use hearing aids. More than 50% of the users are either rejecting them or not satisfied with the hearing aid performance because of lack of training. In addition, There are also about 18 million children with central auditory processing disorders. In addition, patients with tinnitus and or brain injuries with auditory and speech involvements are candidates for auditory training programs. Moreover, these training programs are also appropriate for normal hearing subjects for improving listening and other auditory skills.

The auditory system consists of peripheral organs, afferent and efferent neural tracts. In addition, this system is connected directly or indirectly to extra-auditory structures that may modulate the auditory system's activity. Once sound passes through the peripheral part of the auditory system, it activates neural activity that travels along the afferent auditory fibers and through many nuclei and relays before reaching the auditory cortex. From the ear to the brain, spectral, temporal and intensity information contained in the acoustic signal, are represented using place, timing and level codes of neural activity. It was once believed that these physiological patterns/codes were hard wired and resistant to change. However, current research shows that the central auditory system changes as a function of experience and adapts throughout the life span according to the auditory input that is available to the individual. In case of hearing loss, when sound is reintroduced to the auditory system—modified sensory maps, synaptic alterations and neurochemical changes can be seen following periods of auditory deprivation and auditory stimulation.

Training programs in any field take advantage of the brain's “plasticity capabilities”; i.e., the capacity to change as a function of experience. According to this principle, exercise and auditory training may be designed to activate the brain in ways that help remediate perceptual and/or cognitive functioning.

The term “perceptual learning” can be defined as a process that causes relatively long-lasting changes to an organism's perceptual system in a way that improves its ability to respond to its environment. As regards the auditory system, perceptual learning has been described as the process by which a hearing-impaired person learns to make use of acoustic information (unfamiliar sounds) when first fitted with a hearing aid or cochlear implant.

Auditory training programs are designed to improve the ability to perceive auditory events through repetitive listening exercises. Two kinds of training methods are recognized:
The first is the analytic approach, that emphasized the acoustic content (spectral, temporal and intensity cues) of the signal, and the individual is asked to identify or discriminate sounds that differ acoustically (“bottom up” processing).
The second approach involves synthetic training that is designed to improve perception by enhancing a person's ability to attend to, integrate and use contextual information (“top-down” processing).

Both strategies are currently used in clinics with a pragmatic approach to train the listener to control communication variables such as the level of voice, improving speech/noise ratio (shutting or reducing the TV loudspeaker); or asking guiding questions related to a specified topic in order to obtain the necessary information via audition and understanding the content of the speech.

Studies on animal models have demonstrated that training can alter the physiological representation of sound. Animals that were exposed to a conditioning paradigm showed an increase in neural response magnitude to the conditioning stimulus frequency and a reduction in response magnitude for non-trained frequencies.

Currently, the entire procedure for clinical auditory diagnosis is conducted in clinical sound proof settings and in a single session. This procedure can never predict the actual auditory function in real life situations (competing noises, different hours, different acoustic environments, etc.) There are only a few training software programs in the market. However, these programs do not include any diagnosis procedure, do not relate to the specific problem or deficits of the patient, and are limited to a small number of auditory skills. The current training programs are not specific to the patient's auditory deficit, and thus are not efficient for specific auditory problems. Furthermore, the current software programs are not suitable for patients using hearing aids, and are not adapted to the degree of hearing loss. Thus, patients with hearing loss with or without hearing aids cannot use these systems. In addition, since current programs use free-field stimulation, binaural hearing can not be implemented.

General background information may be found in the following references:

Bakin J. S and Weinberger N. M (1990) Classic conditioning induces CS-specific receptive field plasticity in the auditory cortex of guinea pig. Brain Research 536(1-2):271-286.

Eggermont J. J (1990) The correlation brain: Theory and experiment in neutralinteraction (studies of brain function). New York: Springer-Verlag.

Irvine D. R., Rajan R. and Brown M. (2001) Injury and use-related plasticity in adult auditory system. Audiology and Neuro-Otology 6(4):192-195.

Robinson K. and Summerfield A. Q (1996) Adult auditory learning and training. Ear and Hearing 17(Suppl.):51S-65S.

Sweetow R. and Palmer C. V (2005) Efficacy of individual auditory training in adults: A systematic-review of the evidence. Journal of the American Academy of audiology 16(7):494-504

Watson C. S (1991) auditory perceptual learning and the cochlear implant. American Journal of Otology 12 Suppl:73-79.

Robinson K, Summerfield A Q.: Adult auditory learning and training.: Ear Hear. 1996 June; 17(3 Suppl):51S-65S.

Stecker G C, Bowman G A, Yund E W, Herron T J, Roup C M, Woods D L.:

Shemesh R. : Psychoacoustic tests for central auditory processing: Normative data. J. of Basic & Clinical Physiology and Pharmacology. 2008 19 (3-4) 249-259.

### SUMMARY OF THE INVENTION

Auditory training is an essential tool for habilitation and rehabilitation of communication skills. The training improves speech intelligibility, binaural hearing, localization and auditory cognition, especially in competing and multi-tasks environments. The specific feature of the auditory dysfunction and the specificity of the training are highly important in achieving improvement in the auditory skills. Patients with hearing loss and other auditory symptoms, subjects with normal audiogram but with central auditory processing disorders, patients after brain-injuries are strong candidates for auditory training programs.

Currently, there are no systems that include both diagnostic and treatment tools. All current systems use general training without any specificity to the individual subject's problem and are not compatible for hearing aid users. Moreover, no system is available for children.

The present invention relates generally to the field of auditory skills improvement and, more specifically, to improving auditory perception using a system that screens, diagnoses and trains the auditory system accordingly. The system may be compatible for hearing aid users and for children. The training is specific to the auditory profile of the individual patient with adaptive task difficulties. To monitor and control the training, the system transfers specific data to a control-center at a remote server location. The system remediates and improves the auditory skills including speech understanding and communication skills.

In contrast to the limitations of methods and systems of the art, the current invention, screens and diagnoses more specifically the auditory deficit of the patient and forms an appropriate and specific treatment for children and adult with or without hearing aids. Moreover, by using various paradigms and approaches, binaural hearing may be also trained.

Additionally, in contrast to methods and system of the art that record and diagnose the patient's status only once or a few limited times while the patient is examined at the examination location, the current invention allows multiple testing at plurality of different times, different locations, at different mental and physical conditions such as fatigue, relaxation, tension; etc.; and at different realistic environmental conditions. Generally, the realistic testing and training conditions match the day to day hearing and comprehension challenges the trainee faces, and thus advantageously train the user to face and overcome these challenges.

According to one aspect of the current invention a method for auditory diagnosis and training is provided, the method comprising the step of: in a first stage, providing an automatic set up software capable of: registering a user; identifying the user's general auditory problem based on user's response to a general anamnesis questioner; prepare and present to said user a specific questioner based on said identified general auditory problem; prepare and present to said user a specific battery of hearing tests and auditory processing tests based on said user's response to said specific questioner; and set for the user initial training parameters based on results of said battery of hearing tests; and in a second stage, providing an automatic training software capable of: using said initial training parameters to prepare and present the user with a training session comprising a battery of auditory challenges; testing said user's progress due to said training session; updating the training parameters in response to said user's progress; and repeat the entire second stage using said updated training parameters.

In some embodiments the automatic set up software is capable of collecting demographic data from said user.

In some embodiments the battery of auditory challenges comprises of general training and user-specific training.

In some embodiments the method further comprises reporting said user's progress to a remote server location.

In some embodiments, the automatic training software is further capable of receiving updated training parameter computed by said remote server in response to the user's progress report.

In some embodiments the automatic training software is further capable of: preparing and presenting said user with a pre-training test based on training parameters of previous training session; and updating training parameter for the training session based on results of said pre-training test.

In some embodiments the method further comprises providing said user feedback report based on said user's progress.

In some embodiments the user feedback report comprises a plurality of test results, each test result comprises: test title, lower normal range limit; higher normal range limit; and results of last test.

In some embodiments the user feedback report comprises a plurality of test results, each test result further comprises: at least one of: test result history; and training goal.

In some embodiments the automatic set up software and said automatic training software are capable of being executed outside a medical facility.

In some embodiments the automatic training software are capable of being executed on a pocket size device.

In some embodiments automatic set up software and said automatic training software are capable of being executed on different computing platforms.

In some embodiments automatic set up software and said automatic training software are capable of being executed at different locations and different times

Another aspect of the current invention is to provide a system for auditory training comprising: a remote server in communication with a local processor; at least one audio signal transducer, receiving audio information from said local processor and converting said audio information to audio signals, wherein said local processor is configured to execute an automatic training software capable of: using training parameters to prepare and present the user with a training session comprising a battery of auditory challenges; testing said user's progress due to said training session; and updating the training parameters in response to said user's progress.

In some embodiments the local processor is selected from a group of computing devices such as: a personal computer; a mobile phone; a personal digital assistant; a pocket size multimedia device; a hearing aid interface device; a hearing aid; and an auditory implant interface device.

In some embodiments the audio signal transducer is selected from a group consisting of: a speaker; an earphone set; a hearing aid; and an auditory implant.

In some embodiments the automatic training software is further capable of reporting said user's progress to said remote server.

In some embodiments the remote server is capable of updating training parameters used by said local processor in response to said reported user's progress.

In some embodiments the local processor is configured to execute an automatic set up software capable of: registering a user; collecting demographic data from said user; identifying the user's general auditory problem based on user's response to a general anamnesis questioner; prepare and present to said user a specific questioner based on said identified general auditory problem; prepare and present to said user a specific battery of hearing tests and auditory processing tests based on said user's response to said specific questioner; and set for the user initial training parameters based on results of said battery of hearing tests.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a schematically illustrates a system for auditory diagnosis and training in accordance with an exemplary embodiment of the present invention.

FIG. 1b schematically illustrates a system for auditory diagnosis and training in accordance with another exemplary embodiment of the present invention.

FIG. 1c schematically illustrates a system for auditory diagnosis and training in accordance with another exemplary embodiment of the present invention.

FIG. 1d schematically illustrates a system for auditory diagnosis and training in accordance with another exemplary embodiment of the present invention.

FIG. 1e schematically illustrates a system for auditory diagnosis and training in accordance with yet another exemplary embodiment of the present invention.

FIG. 2a illustrates a block diagram of the setup stage of a method of auditory diagnosis and training in accordance with an exemplary embodiment of the present invention.

FIG. 2b illustrates a block diagram of the training stage of a method of auditory diagnosis and training in accordance with an exemplary embodiment of the present invention.

FIG. 3 schematically depicts display of training results according to an exemplary embodiment of the current invention.

### DESCRIPTION OF PREFERRED EMBODIMENTS

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The terms “comprises”, “comprising”, “includes”, “including”, and “having” together with their conjugates mean “including but not limited to”.

The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

In discussion of the various figures described herein below, like numbers refer to like parts. The drawings are generally not to scale. For clarity, non-essential elements were omitted from some of the drawing.

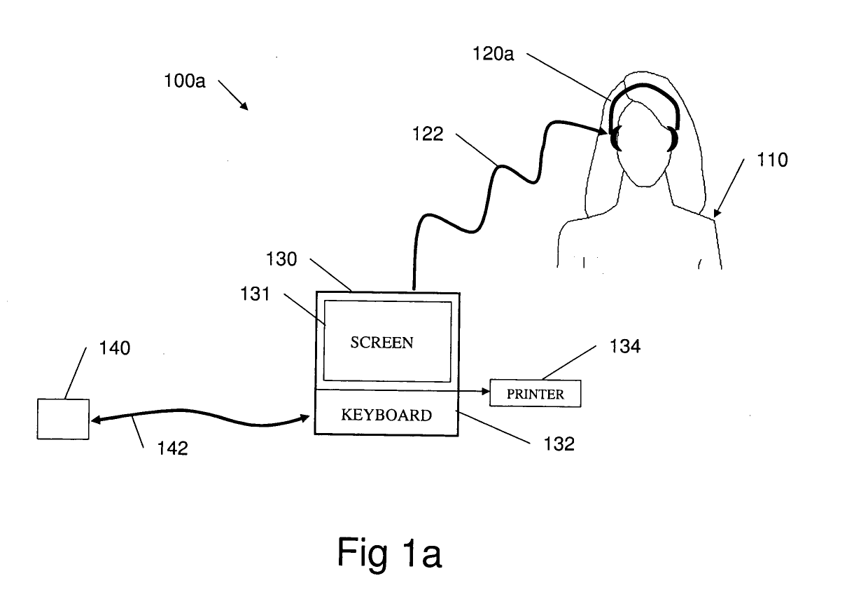

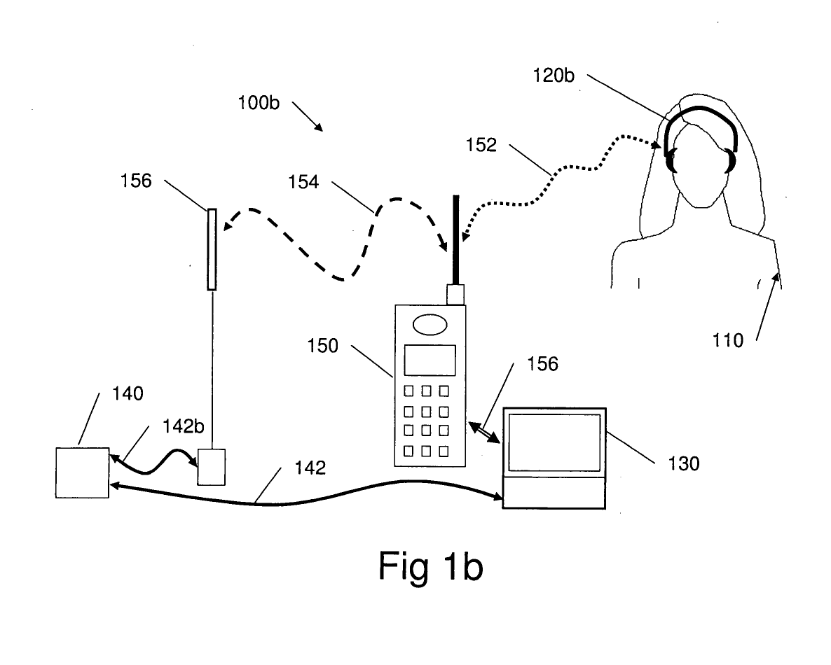

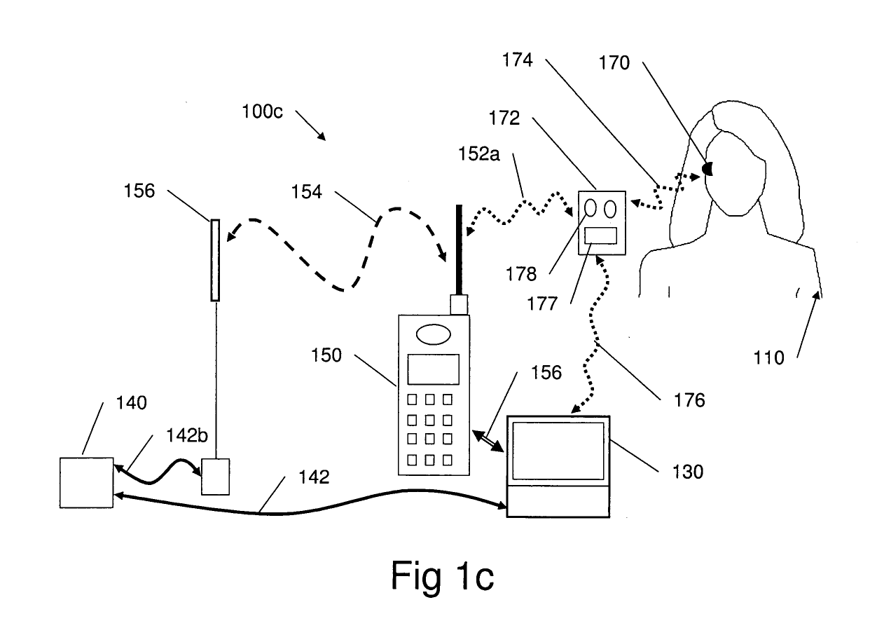

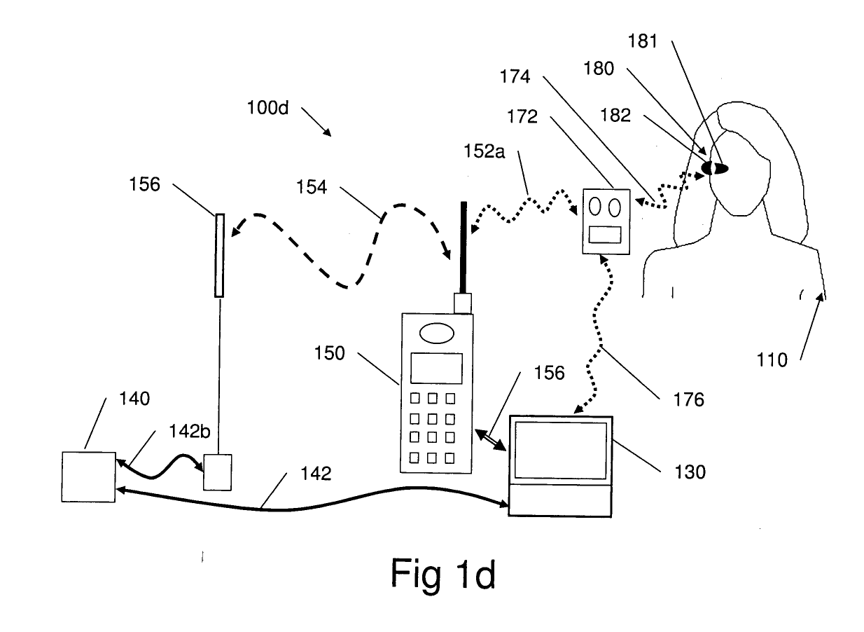

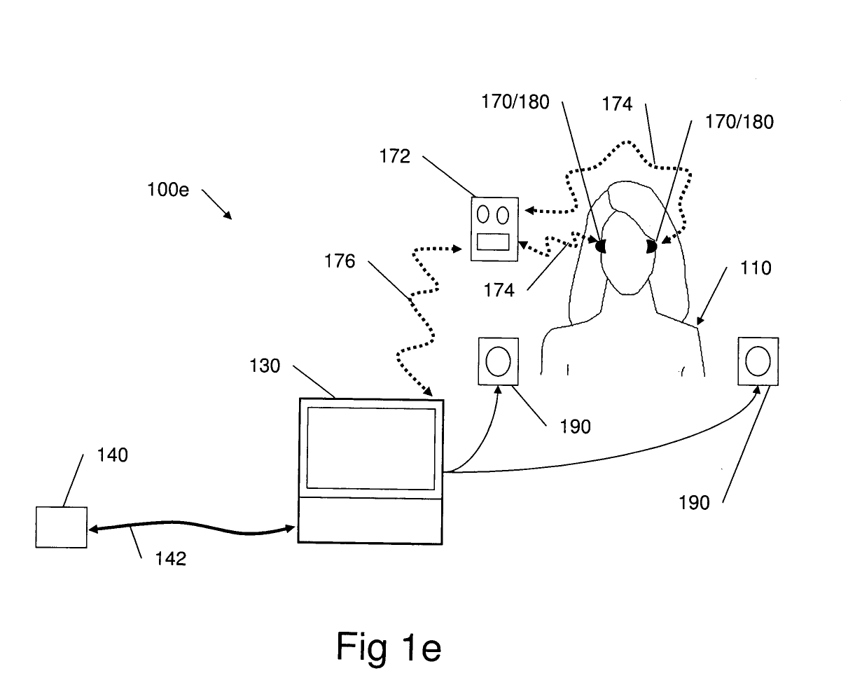

Refereeing to the figures, FIG. 1a schematically illustrates a system 100a for auditory diagnosis and training in accordance with an exemplary embodiment of the present invention.

System 100a uses a personal computer (PC) 130 such as a desktop, laptop or notebook or notepad computer for executing the diagnostic and training program and earphone set 120a for audio input to patient 110.

In the depicted exemplary embodiment, earphone set 120a is standard earphones, connected with cord 122 to the PC 130. However, preferably earphone set 120a is a specifically calibrated set supplied with the diagnostic and training program. In a preferred embodiment of the invention, earphone set 120a is a USB earphone set as will be shown below. Optionally, earphone set 120a is a wireless earphone set and cord 122 is replaced with wireless communication channel such as Infrared, Wi-Fi or Bluetooth short range communication channel. In the drawings, earphones 120 are depicted as “over the ear” earphones, however it should be realized that “button earphones” inserted into the outer ear may be used. It also should be noted that in some cases, for example total hearing loss in one ear, a single earphone may replace the earphone set 120.

PC 130 comprises a processor, volatile and nonvolatile memory, input device such as keyboard 132, display such as screen 131 and optionally printer 134.

PC 130 is capable of, connecting to a remote server 140 via long range communication channel 142 such as phone line or the Internet. Remote server 140 is capable of exchanging information with PC 130. For example, remote server 140 may load or update the diagnostic and training program. Additionally, remote server 140 receives results of diagnostic tests and training results performed by user 110, analyses the results and in response to these results sends training parameters to the PC 130. Additionally, server 140 monitors the training compliance of user 110 and provides billing services to the operators of the diagnostic and training program.

FIG. 1b schematically illustrates a system 100b for auditory diagnosis and training in accordance with an exemplary embodiment of the present invention.

System 100b uses a mobile device such as cellular phone 150 for executing the diagnostic and training program and earphone set 120b for audio input to patient 110. Mobile device 150 may be a cellular phone, a Personal Digital Assistant (PDA) device or a multimedia mobile device such as iPod, etc. Alternatively, mobile device 150 is a proprietary pocket size device comprising: power source such as battery; processor with its volatile and non-volatile memory; display; input means such as keypad, touch-screen or joystick; and corded or wireless connection to an earphone. Diagnostic and training program may be loaded into the mobile device 150 and be executed on its processor similarly to the manner in which applications and games are loaded and executed. Mobile device 150 comprises input devices such as keys or touch screen and a display.

In the depicted exemplary embodiment, earphone set 120b is standard corded or wireless earphones, connected via short range communication link 152 to mobile device 150.

Earphone set 120b may be supplied with the mobile device, however, preferably earphone set 120b is a specifically calibrated set supplied with the diagnostic and training program or selected from a list of approved earphone sets and purchased separately. Alternatively, earphone set 120b is supplied together with the mobile device 150 or purchased by the user and is calibrated in a calibration station operated by the supplier of the diagnostic and training program. Alternatively, the volume of sound in the earphones is determined subjectively by the user, or in response to results of hearing tests performed on the user using the earphone set.

Optionally, different volume setting is used for each rear. In a preferred embodiment of the invention, mobile device 150 is preferably capable of communicating remote server 140 using cellular network having a cellular base station 152 over long range RF channel 154 and land communication links 142b.

In some embodiments, earphones 120a or 120b provides some insulation against environmental noise. Earphones 120a or 120b may comprise passive noise redacting acoustic insulation or may comprise active noise cancelation subsystem as known in the art.

By using a mobile device 150, user 110 may execute the diagnostic and training to program anywhere and anytime, for example outdoors or on the go, without the need of having a PC or power outlet nearby. This freedom greatly increases the ease and frequency of training as training can be done while traveling, etc. Specifically, passive training which comprises auditory input to the user and does not require interactive, immediate or frequent response may be done.

In some embodiments system 100b additionally comprises a PC 130 with its optional peripherals. Optional PC 130 may be periodically used to view or print training progress.

Additionally or alternatively, mobile device 150 may communicate with remote server 140 by interfacing to PC 130 using optional corded or short range wireless communication link 156. In the cases where mobile device 150 is a multimedia device lacking cellular capabilities, communication with remote server 140 may be done via PC 130 and link 142.

In some embodiments, earphones 120a or 120b may also be equipped with a microphone or microphones, placed inside and/or outside the earpiece for monitoring noise and audio levels.

FIG. 1c schematically illustrates a system 100c for auditory diagnosis and training in accordance with another exemplary embodiment of the present invention.

In the embodiment depicted in FIG. 1c, earphone set 120a or 120b are replaced with Hearing Aid (HA) 170. For users needing HA for their daily routines, using HA for training may be advantageous as training is performed in the same environment as their daily use. Although one HA is seen in this figure, both ears may be fitted with HA's.

In some embodiment, system 100a or 100b may be used wherein one or two HA's are used under earphones 120a or 120b. In these embodiments, simple HA's capable of only amplification and optionally filtering may be used.

In other embodiments, sophisticated EA's may be used, For example, sophisticated EA 170 is in communication with interface device 172 via a preferably short range wireless link 174. Short range wireless link 174 is often a proprietary communication protocol designed by the HA manufacturer, but may optionally be a commonly used protocol. Sophisticated EA 170 is capable of receiving audio information from interface device 172 and converts them to sound signals to be heard by user 110.

Interface device 172 is commonly used for testing and configuring HA 170 and for adopting HA 170 to the specific needs of user 110. According to an exemplary embodiment of the current invention, interface device 172 is in communication with at least one of mobile device 150 or PC 130 via links 152a and 176 respectively.

In one embodiment, diagnostic and training program is executed in one of PC 130 or mobile device 150. In these cases interface device 172 is used by the program to relay audio signals from PC 130 or mobile device 150 to HA 170. As HA 170 comprises a microphone, it may be used for transmitting signal indicative of the environmental noise present during the training to the PC or mobile device and be used by the diagnostic and training program for adjusting training and testing parameters. These signals may be used by the program to adjust the training or testing parameters, for example to increase the audio volume in noisy environment, to suspend or to repeat a training session or test if noise level was excessive, or to present the user with challenges compatible with the existing noise level.

When used under earphones 120, microphone in HA 170 may be used for measuring and calibrating the audio output of earphones 120 as well as for noise monitoring.

If HA 170 is capable of directly communicate with any of mobile device 150 or PC 130, interface device 172 may be omitted.

Interface device 172 is preferably pocket sized and may comprise a rudimentary display 177 such as alphanumeric LCD display 177 or LED indicator. Interface device 172 preferably comprises user input devises such as pushbuttons 178. During testing and active training, user may use input devices 178 to indicate his response to training or testing challenges. Alternatively, the user uses keyboard 132 or input devices on mobile device 150 to provide his response to training or testing challenges.

Interface device 172 may comprise a processor and memory capable of executing at least parts of the testing and training program. For example, a new training routine may be uploaded from remote server 140 to the interface device 172 via PC 130 or mobile device 150 and be executed within interface device 172. In this embodiment, the user may only need to carry the small interface device 172 and wear his HA. Responses to testing and training challenges may be stored in memory of interface device 172. These results may be reported to the PC, mobile device or the remote server and used for adjusting training parameters and for providing the user with progress reports.

In some embodiments, sophisticated HA 170 comprise a processor and memory and capable of executing at least some of the diagnostic and training program. For example, a new training routine may be uploaded from remote server 140 to HA 170 via the interface device 172 or directly via PC 130 or mobile device 150 and be executed within HA 170. In this embodiment, the user may only need to wear his HA. Passive training, in which no user response is required, may be executed by HA having no user input. Alternatively, voice or sound command *such as clapping hands or clicking fingers, etc) may be used by the user to provide response to testing and training challenges. As HA 170 does not comprise indicators such as a display or LEDs, voice or sound feedback may be provided by HA 170 to inform the user if he/she met a test challenge. Responses to testing and training challenges may be stored in memory within HA 170. These results may be reported to the PC, mobile device or the remote server and used for adjusting training parameters and for providing the user with progress reports.

FIG. 1d schematically illustrates system 100d for auditory diagnosis and training in accordance with another exemplary embodiment of the present invention.

In the embodiment depicted in FIG. 1d, HA's 170 are replaced with Auditory Implants (AI) 180 such as cochlear implant; middle ear implant; or brain implants. AI's are used by user suffering from total or near total hearing loss and may be implanted in one or both ears. AI 180 comprises an implant 181 and external part 182 as known in the art.

Generally, the external part 182 comprises a processor and thus AI 180 may be adopted to act as a sophisticated HA described in FIG. 1c.

FIG. 1e schematically illustrates system 100e for auditory diagnosis and training in accordance with yet another exemplary embodiment of the present invention.

In the embodiment of FIG. 1e, HA's 170 and/or AI's 180 are used by user 110 during testing and/or training session.

PC 130 is connected to at least one and preferably a pair of speakers 190.

According to this exemplary embodiment, speakers 190 provide audio signals for the testing and training while HA's 170 are used for amplification and filtering. Similarly, AI's 180 used for providing the user with auditory signals in the case of user's total or near total hearing loss.

Optionally, microphones in HA's 170 or AI 180 provide sound level calibration to the testing and training program executed on PC 130.

It should be noted that in all the abovementioned embodiments, devices and systems are provided with necessary parts and subsystems as known in the art which may have been omitted from the disclosure for clarity. For example, devices may comprise: power sources such as batteries, rechargeable batteries or power supplies. Processors may comprise volatile and non-volatile memories. Microphones, earphones and speakers may comprise amplifiers and filters; etc.

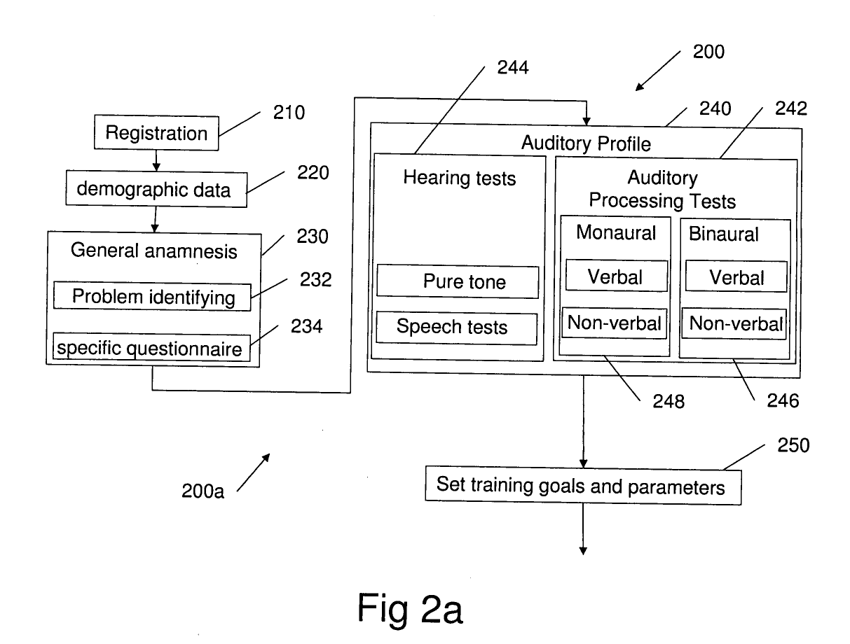

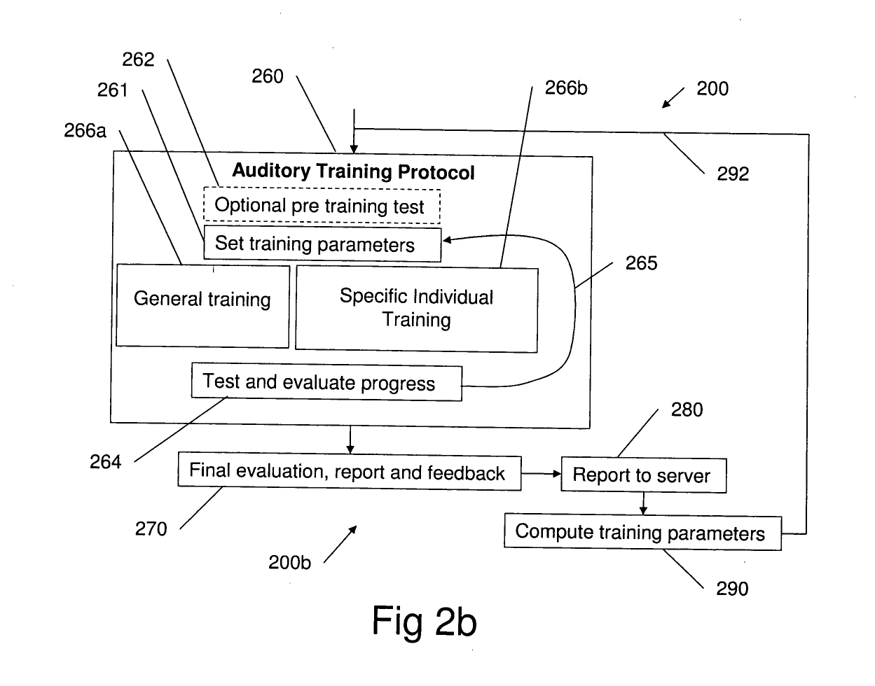

FIG. 2a illustrates a block diagram of setup stage 200a of a method 200 or auditory diagnosis and training in accordance with an exemplary embodiment of the present invention.

In the registration stage 210, the patient is register in the system and preferably supplied with a code for identification and for upload program or creating new library.

Registration stage 210 may be done in a participating clinics or doctor's office, by phone or by logging to the program provider's Internet website.

At the registration stage, the user gives his identification details and preferably provides means of payment for participating in the diagnostic and/or training program. Payment for participating in the training program may be in the form of a one-time fee for purchasing the software and the optional hardware needed for the diagnostic and training program. Additionally or alternatively, fee may comprise a per-use fee for the training, a monthly or annual fee, a fee based on performance improvement, etc.

After registration, optional demographic data collection stage 220 may be used for collecting demographic data of the patient is collected, for example age, gender, to profession, academic background etc. In some cases, for example if this information is already available (for example at the treating clinics) or if the patient prefers to guard his privacy, this stage may be omitted. Preferably this stage is automatic and the user enters the data by answering an electronic questioner by selecting the relevant reply from a multiple choice menu.

General anamnesis stage 230 is then used for obtaining general anamnesis procedure including medical history, risk factors for hearing problems, usage of hearing aids and assisting devices etc.

Preferably, in the first step 232 of stage 230, the specific problem (or problems) of the patient is (are) identified from the patient's response to general questions about his condition and difficulties.

In some cases this information may already be available (for example at the treating clinics), and this stage may be omitted. Preferably this stage is automatic and the user enters the data by answering an electronic questioner by selecting the relevant reply from a multiple choice menu. In clinic setting this stage may be assisted by trained personnel such as a paramedics nurse or a doctor.

Preferably, following the problem identifying stage 232, in the second step 234 of general anamnesis stage 230, the system inquires about specific auditory problems (identified in the stage 232 above) and presents the user a specific questionnaires regarding: tinnitus; communication skills, hyperacusis, stress level, and dizziness. In clinic setting this stage may be assisted by trained personnel such as a paramedics nurse or a doctor.

In the following auditory profile stage 240 (Diagnosis Phase), auditory profile of the patient is determined. This stage may comprise hearing test 244 and an auditory processing battery of tests 242.

Hearing test 244 may comprises tests for pure tones, noises and speech, and may be monaural and binaural.

Auditory processing battery of tests 242 preferably comprises, both of monaural 248 and binaural 246 presentations both for verbal and non verbal stimuli. More details of stage IV are given below.

Based on the patient's auditory profile created in diagnostic stage 240, the system creates 250 an individual training protocol that comprise general training targeted individual training and optionally set training goals.

FIG. 2b illustrates a block diagram of training stage 200b of a method 200 of auditory diagnosis and training in accordance with an exemplary embodiment of the present invention.

After setup stage 200a, the patient may enter training stage 200b. Training stage 200b comprises a plurality of auditory training protocol 260. Optionally, initial parameters 261 for the training session are determined by one or combination of some or all of: the auditory profile as determined in setup stage 200a, by the results of previous training session (if any), and by a results from an optional pre-training test 262.

In each task difficulty level, the patient preferably gets feedback information 270 relate to his/her performance.

The training program comprising general training 266a and specific individual training 266b is preferably is automatic and self adaptive 265 changing tasks and difficulty according to the patient's auditory state as determined by a progress evaluation 264. The system repeatedly checks the patient's current auditory profile and compares it to its pre-training status and thus computes and monitors the changes in auditory function.

Periodically, the system reports 280 the training results to the remote server that may use these results to compute 290 new training parameters and update 292 the training program. However, a training session may commence without reporting results of previous session to the server. If a session is interrupted, session may resume later or a new session may be started afresh.

In some embodiments, server 140 automatically process training and or testing reports. Optionally, train personnel at the remote server monitor the training progress and alert or inform the patient of any abnormalities or provide him with advice or assistance. Optionally remote server provides the patient with a progress report, optionally in addition to progress reports produced locally at the patient's location.

More details of this stage are given below.

Some details of Stage 240: Set up the Auditory Profile

A purpose of the auditory profile is to have an assessment of the patient's peripheral and central auditory function to verbal and non-verbal auditory stimuli presented monaurally and binaurally. The level of the auditory presentation may be based on the patient's hearing threshold.

Non-Verbal Tests

The non-verbal tests comprise at least some of the following:

A. Loudness perception:
1. Finding the loudness dynamic range for pure tone, noise and speech stimuli. In “non-verbal” tests; speech stimuli refers only to the comfortable and uncomfortable level not to the understanding of speech
2. Just Noticeable Difference (JND): Assessing the minimal level change required for a different percept in a number of frequencies.

3. Loudness Integration: Assessing the loudness perception as a function of the time duration of the stimuli. As the duration shortens, the threshold increases.

B. Pitch Perception:
1. Pitch discrimination—JND for frequency; Pitch Integration—assessing the pitch perception as a function of the duration of the stimuli.
2. Pitch modulation—JND for modulation rate.
3. Pitch pattern sequence—Discrimination and ordering pitch signal in a sequential pattern.
4. Missing fundamental frequency—The auditory system's success in detecting the fundamental frequency in the complex sound even in the absence of this frequency.
5. Binaural beats—The beats are heard in the simultaneous presentation of different but close frequencies to each ear.

C. Temporal Resolution:
1. Gap detection—Detection of the minimal gap separating consecutive stimuli.

D. Masking Tasks:
1. Masking Level Difference (MLD)—The noise is suppressed by the central auditory system when presented binaurally with pure tones or speech signal resulting in better thresholds of the pure tones
2. Central Masking—The threshold of the target signal is elevated in the presence of contra lateral noise or a competing similar signal. This phenomenon depends on an intact central auditory masking system.
3. Temporal Masking; Backward Masking—Signal is suppressed by a preceding (3-5 msec) masking noise.

E: Directional Hearing:
1. Lateralization processing—The ability of the auditory system to localize the auditory signal over the head space based on interaural differences of time and level.

Verbal Tests:

The verbal tests comprise at least some of the following:
1. Speech in Noise Tests—Measuring the threshold of a speech signal (for example detection of 50% of presentations) in the presence of noise (Signal to Noise Ratio). The speech stimuli may include two-syllable words (meaningful and non meaningful words) or sentences with high and low semantic redundancy.
2. Simulate Rapid Speech—Detection of Speech thresholds for rapid sentences.
3. Competing sentences—Threshold for detection of words in competing sentences presented simultaneously to both ears. The competence effect may include level differences, pitch differences, masking noises and stimuli (reverse speech; babble or cafeteria speech etc.).
4. Dichotic Listening—Simultaneous presentation of different signals to both ears. The signals may include digits, words or sentences. The task is scored according to discrimination of the target stimuli.
5. Rapidly alternating speech perception—Binaural re-synthesis sample from the rapidly alternating speech perception.
6. Filtered speech—Simultaneous Presentation of similar words to ears, one with High Pass and the other with Low Pass filtering. The task is to identify the target word.

Cognitive Auditory Tasks:

Cognitive Auditory Tasks are aimed to assess cognitive functions by measuring reaction times, discrimination scores; errors and types of errors. These may comprise:
1. Auditory Oddball Paradigm—The subject is asked to quickly and accurately discriminate a target in the presence of non-target stimuli. The discriminative measure may include, loudness differences, pitch changes, duration, meaningful and non meaningful words, words of different classes (animate-inanimate, male-female).
2. Selective and focused auditory attention tasks—The subject is asked to report a specific target in the presence of competing non-target stimuli presented to the other ear. The discriminative measure may include both contextual and physical differences.
3. Auditory short term memory tasks—The subject hears set of digit numbers and asked to repeat them. Score is related to the number of correct digits repeated.

Cross Modal Interaction
1. McGurK Effect—The interaction between the visual and auditory processing result in changes in auditory perception.

Some details of Stage 260: Auditory training

Optionally, the user undergoes before 262 the training and after 264 the training sessions a battery of auditory diagnostic tests. These tests are created according to of the following principles:
1. The diagnostic test are preferably reliable and reproducible
2. The diagnostic test preferably do not exhibit strong and rapid learning effects, thus repeated tests preferably remained unbiased.
3. The diagnostic test are preferably be efficient because of limited testing time
4. The procedure of the test should is well described and involve a short practice period.
5. The diagnostic test is preferably applicable to a wide range of severity and types of hearing disorders.
6. The test diagnostic preferably has relevance to the subject and his/her auditory problems.

Principles of training 266a and 266b:

Based on the subject's pre-training 262 score, the training program automatically evoke a training session 266a and 266b that preferably comprise of increasing difficulty and provides feedback information to the subject both auditory and visually. The starting level is preferably approximately one Standard Deviation (SD) below the patient's pre-training score 262, and preferably includes specific 266b and general 266a tasks. The training is adaptive (265 and 292) and interactive and preferably always presenting tasks that are challenging but not frustrating. The training preferably includes immediate visual and auditory feedback to maximize perceptual learning.

Each task preferably begins with instructions to the patient presented through the earphones and/or on the screen, optionally followed by demonstrations of the tasks (forward practice). However, an experienced patient may skip these demonstration and instruction steps.

The training relies on two basic channels of brain information processing:
One is the “bottom up” processing including degraded speech exercises, rapid speech and compressed time speech and competing speaker.
The second channel is “top down”, consisting of language and contextual tasks, e.g., the target word task. The listener is visually presented with a target word. A sentence is played containing the target word. The listener is asked to identify the word that precedes the target word. If the selected word is correct for two tasks in a row, the next task becomes more difficult. For example the sentence may be presented before the target word is identified, thus making the listener retain the entire sentence into short term memory. The task can be made more difficult by presenting two target word and two sentences, and so on.

In addition to training of the bottom up and top down skills, the subject will be subjected to helpful cognitive strategies that will provide the patients realistic expectations and keep them motivated.

According to the patients' individual auditory profile, they are trained with a specific program aimed to improve their auditory performance. The training is preferably daily, lasting for example 20 minutes a day for 6 weeks. Following this period, the patient's auditory profile is checked again.

Preferably, some or all of: the results of the auditory tests before and after training, the duration, dates and time of the individual training are submitted to a control center at remote server location 140 automatically or by user's command.

In addition to monitoring users' progress and providing billing services, server 140 may perform statistical and individual analysis of training efficacy and improve training parameters choice. Server 140 may provide recommendations and instructions directly to the patient or to the treating doctor. Specifically, the system may be used as early alarm sensitive to any deterioration in user's performance. For monitoring purposes, infrequent testing may be sufficient. For example, the system, locally or by command from server, may prompt the user to perform a periodic test, for example one weekly or monthly, and based on the test result recommend one of: continue with scheduled monitoring tests if the test result were compatible with previous tests; start a new set of training if test results deteriorate slightly; or suggest that the patient would seek professional medical help if large deterioration was detected or a new auditory deficiency was detected. Server 140 may also monitor patient's compliance with the training program. For example, if the local processor (PC, mobile device, etc) were inactive, turned off, did not make contact with the server, or reported absence, partial, infrequent or incomplete training, server 140 may prompt the user to comply with the program by mail, e-mail, voice message, or having a person contacting the user. Similar notification may be issued to the caretaker or the doctor of the patient.

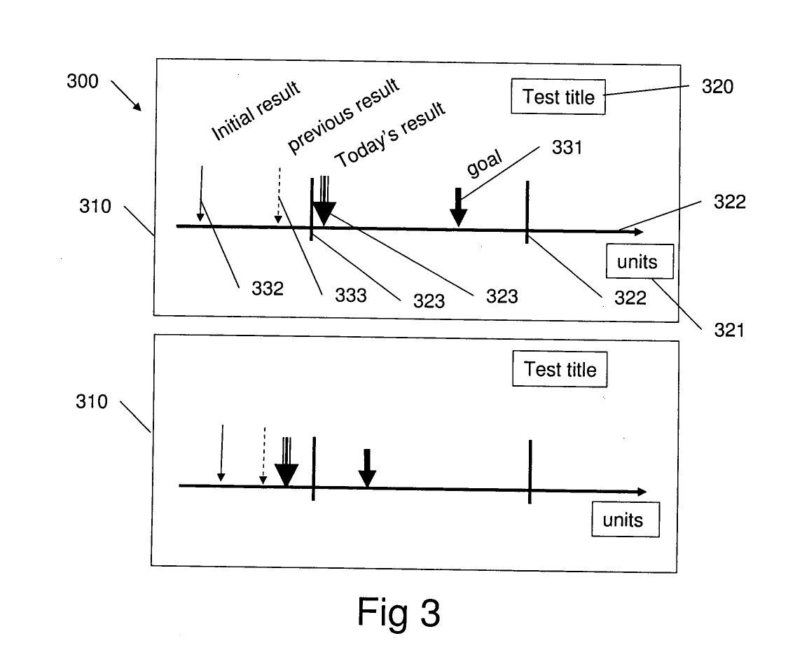

FIG. 3 schematically depicts display of training results report 300 according to an exemplary embodiment of the current invention.

According to an exemplary embodiment of the invention, reports of training results 300 are preferably displayed such that they provide clear and easy to understand feedback to the user.

Training results may be periodically mailed to the user by the remote server 140. Alternatively or additionally, report 300 may be computed by PC 130 and displayed or printed by the user. Alternatively or additionally, report 300 may be computed and displayed mobile device 150. In some embodiments at least some results are available to the user after each training session.

Report 300 comprises at least one and preferably a plurality of test results 310.

Test result 310 comprises a title 320 detailing the type of test or the auditory parameter tested. Optionally date of the test is also displayed for the entire report and optionally for each test type (if test types were performed on different dates).

Preferably, the units 321 such as dB or percents are displayed near a preferably scaled arrow 322 having arrow head pointing to better results. Normal accepted range relevant to the test type and the population of similar users is marked by upper normal limit 323 and lower normal limit 324, optionally with the relevant values of these limits.

The last measured test result 324 is marked in relation to the upper and lower normal range limits 322 and 322, optionally with its numerical value.

Optionally, additional information is displayed, preferably using clear and distinct marks. The additional information may be one or several of: Training goal 331; initial test results obtained before the training had started 332 and results obtained in the previous test 333.

Candidates for acquiring the training system may come from hearing professionals, ENT doctors, Neurologists, Aural rehabilitation centers, hearing aid companies and dispensers, cochlear implants and hearing assistive device companies, educational and special education teachers. In addition, any one interested in improving auditory skills (better understanding in noise and in competing signals, enhancement of auditory attention).

In addition, the diagnostic part of the method, together with system 100a or 100b may be used as automatic screening station to be used in clinics, working places, elderly homes, educational establishment and the likes to screen for auditory problems efficiently and cheaply and with minimal or no need for highly trained personnel.

Studies were conducted to test the efficiency of the method of auditory training.

The aim of the first study was to evaluate the effects of auditory training on speech understating in background noise in 20 normal hearing subjects. The results showed that binaural or unilateral training increased significantly the understanding from baseline of about 60% of speech understanding to monosyllable words or sentences, to more than 85% after training. Furthermore, similar increase in understanding was found also in new speech stimuli that he has never been trained before .

In the second study 36 elderly hearing impaired subjects (ages: 64-88) were fitted with binaural hearing aids, either simultaneously or sequentially, and auditory training was applied while the subjects were wearing their personal hearing aids. Significant and large improvement was found in dichotic speech scores and in the speech in noise perception in the unaided condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for auditory diagnosis and training comprising the step of:
a. providing an automatic set up software capable of:
registering a user;
identifying the user's general auditory problem based on user's response to a general anamnesis questioner;
prepare and present to said user a specific questioner based on said identified general auditory problem;
prepare and present to said user a specific battery of hearing tests and auditory processing tests based on said user's response to said specific questioner; and
set for the user initial training parameters based on results of said battery of hearing tests;
b. providing an automatic training software capable of:
using said initial training parameters to prepare and present the user with a training session comprising a battery of auditory challenges;
testing said user's progress due to said training session;
updating the training parameters in response to said user's progress; and
repeat the entire step b. using said updated training parameters.

2. The method as claimed in claim 1, wherein said automatic set up software capable of collecting demographic data from said user.

3. The method as claimed in claim 1, wherein said battery of auditory challenges comprises of general training and user-specific training.

4. The method as claimed in claim 1, and further comprising reporting said user's progress to a remote server location.

5. The method as claimed in claim 4, wherein said automatic training software further capable of receiving updated training parameter computed by said remote server in response to said user's progress report.

6. The method as claimed in claim 1, wherein said automatic training software further capable of:
preparing and presenting said user with a pre-training test based on training parameters of previous training session; and
update training parameter for the training session based on results of said pre-training test.

7. The method as claimed in claim 1, and further comprising providing said user feedback report based on said user's progress.

8. The method as claimed in claim 7, wherein said user feedback report comprises a plurality of test results, each test result comprises: test title, lower normal range limit; higher normal range limit; and results of last test.

9. The method as claimed in claim 8, wherein said user feedback report comprises a plurality of test results, each test result further comprises at least one of: test result history; and training goal.

10. The method as claimed in claim 1, wherein said automatic set up software and said automatic training software are capable of being executed outside a medical facility.

11. The method as claimed in claim 10, wherein said automatic training software are capable of being executed on a pocket size device.

12. A system for auditory diagnosis and training comprising:
a remote server in communication with a local processor;
at least one audio signal transducer, receiving audio information from said local processor and converting said audio information to audio signals, wherein said local processor is configured to execute an automatic training software capable of:
using training parameters to prepare and present the user with a training session comprising a battery of auditory challenges;
testing said user's progress due to said training session; and
updating the training parameters in response to said user's progress.

13. The system for auditory diagnosis and training claimed in claim 12, wherein said local processor is selected from a group consisting of: a personal to computer; a mobile phone; a personal digital assistant; a pocket size multimedia device; a hearing aid interface device; a hearing aid; and an auditory implant interface device.

14. The system for auditory diagnosis and training claimed in claim 12, wherein said audio signal transducer is selected from a group consisting of: at least one speaker; and an earphone set.

15. The system for auditory diagnosis and training claimed in claim 12, wherein said audio signal transducer is selected from a group consisting of: at least one hearing aid; and at least one auditory implant.

16. The system for auditory diagnosis and training claimed in claim 12, wherein said automatic training software is further capable of reporting said user's progress to said remote server.

17. The system for auditory diagnosis and training claimed in claim 16, wherein said remote server is capable of updating training parameters used by said local processor in response to said reported user's progress.

18. The system for auditory diagnosis and training claimed in claim 12, wherein said local processor is configured to execute an automatic set up software capable of:
registering a user;
identifying the user's general auditory problem based on user's response to a general anamnesis questioner;
prepare and present to said user a specific questioner based on said identified general auditory problem;
prepare and present to said user a specific battery of hearing tests and auditory processing tests based on said user's response to said specific questioner; and
set for the user initial training parameters based on results of said battery of hearing tests.

19. The method as claimed in claim 12, wherein said automatic set up software capable of collecting demographic data from said user.
